(19) United States
(12) Patent Application Publication
(10) Pub. No.: US 2006/0199224 A1
(43) Pub. Date: Sep. 7, 2006

(54) Parallel identification of antibacterial targets and inhibitors

(75) Inventors: Frank Schmidt, Columbia, VA (US); Larisa Dedkova, Charlottesville, VA (US); Sidney Hecht, Charlottesville, VA (US)

Correspondence Address: JOSEPH C. ZUCCHERO, SUITE 1200, 500 WEST CUMMINGS PARK, WOBURN, MA, 01801, US

(73) Assignee: Pinnacle Pharmaceuticals

(21) Appl. No.: 11/351,902

(22) Filed: Feb. 10, 2006

Related U.S. Application Data
(60) Provisional application No. 60/652,055, filed on Feb. 11, 2005.

(51) Int. Cl. C40B 40/02; C40B 40/04; C12Q 1/18

(52) U.S. Cl. 435/7.1; 435/32

(57) ABSTRACT

The invention relates to methods for the parallel identification of compounds that have antibacterial activity as well as the target gene for that compound. This approach allows for coupled target validation/drug discovery, elucidation of mechanism(s) of antibacterial agents as well as discovery of new antibacterial pharmacophores having the same mechanism of action as existing agents.

### RELATED APPLICATIONS

This application claims the benefit of U.S. Provisional Patent Application Ser. No. 60/652,055, filed on Feb. 11, 2005, the contents of which are incorporated herein by reference in its entirety.

### BACKGROUND OF THE INVENTION

1. Field of the invention

The invention relates to identification of genes essential for bacterial viability and inhibitors of the products of those genes.

2. Summary of the related art

During the past twenty years, the number of completely sequenced bacterial genomes has greatly increased. It was found that bacterial genomes contain more than 2000 genes but current antibiotics target only a small handful of them. The collection of genomic data has provided new insights into the molecular structure of cells, revealing the basic genetic and metabolic elements that support cell viability. Recently, open reading frames (ORFs) predicted from all known genomic sequences have been organized into Clusters of Orthologous Groups (COGs). The COG approach in combination with BLAST (Basic Local Alignment Search Tool) searching of functional ORFs has become a powerful tool in drug discovery. In view of the recent problems with antibiotic resistance there is a need to expand the number of druggable targets.

Three main characteristics are required for antibiotic targets: First, they must be essential for viability or required for cell infection. Second, the bacterial target needs to be significantly different from its mammalian counterpart. Third, it must be present in pathogenic strains. The practical application of this information is the ability to link metabolic activity with its requisite gene target or targets. Once a target has been identified, its overall importance can then be validated through the monitoring of cell survival.

The genomic data is available. The problem lies in the sheer volume of information available, making it hard to use outright. As more and more organisms are sequenced, the problem becomes magnified. The main problems to be solved are how to locate the essential genes needed to make a new drug and how to discover inhibitors of that target. Even if a target gene is identified, to study it requires an inhibitor of its functionality.

### BRIEF SUMMARY OF THE INVENTION

The invention relates to identification of genes essential for bacterial viability and inhibitors of those gene products. In a first aspect, the invention provides a method for the parallel identification of an antibacterial compound from an extract comprising a complex mixture of compounds one or more of which shows antibacterial activity and a target gene for that antibacterial compound. In the method according to this aspect of the invention, bacterial cells comprising a genomic library are contacted with the extract on a plating medium and cells that are resistant to the extract are selected, and the target gene that confers resistance to the extract is identified. The identification of the antibacterial compound in the extract is guided by exposing the sensitive and resistant cells to various fractions of the extract.

In a second aspect, the invention provides a method for using a complex mixture of compounds, one or more of which shows antibacterial activity for the parallel identification of an antibacterial compound and a target gene for that antibacterial compound. In the method according to this aspect of the invention, bacterial cells comprising a genomic library are contacted with the complex mixture of compounds on a plating medium and cells that are resistant to the complex mixture of compounds are selected, and the target gene that confers resistance to the complex mixture of compounds is identified. The complex mixture of compounds is fractionated and the identification of the antibacterial compound in the complex mixture of compounds is guided by exposing the sensitive and resistant cells to various fractions of the complex mixture of compounds.

In a third aspect, the invention provides a method for identifying the mode of action of existing antibacterial agents. In the method according to this aspect of the invention, bacterial cells comprising a genomic library are contacted with one or more known antibacterial agents on a plating medium and cells that are resistant to the antibacterial agent are selected. The target gene that confers resistance to the extract is identified and the activity of the protein or RNA encoded by the target gene is determined.

In a fourth aspect, the invention provides a method for finding antibacterial chemotypes that function by a specific mechanism of action. In the method according to this aspect of the invention, bacterial cells comprising a plasmid comprising a known gene are contacted, on a plating medium, with an extract comprising a complex mixture of compounds one or more of which shows antibacterial activity. The extract is fractionated and the identification of the antibacterial compound in the extract is guided by exposing resistant cells to various fractions of the extract.

### BRIEF DESCRIPTION OF THE DRAWINGS

FIG. 1 is a schematic representation of one embodiment of the methods described herein.

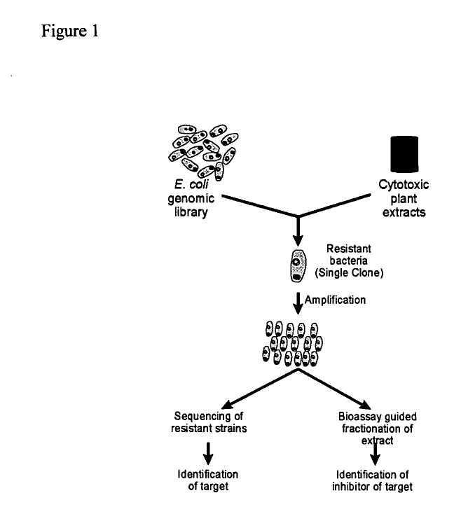

FIG. 2 is a schematic representation of a bioassay-guided fractionation of a crude extract of C010201 (Dysidea sp.).

### DETAILED DESCRIPTION OF THE PREFERRED EMBODIMENTS

The invention relates to methods for the parallel identification of compounds that have antibacterial activity as well as the target gene for that compound. This approach allows for coupled target validation/drug discovery, elucidation of mechanism(s) of antibacterial agents as well as discovery of new antibacterial pharmacophores having the same mechanism of action as existing agents. The patents and publications cited herein reflect the level of knowledge in the art and are hereby incorporated by reference in their entirety. Any conflict between the teachings of these patents and publications and this specification shall be resolved in favor of the latter.

In a first aspect, the invention provides a method for the parallel identification of an antibacterial compound from an extract comprising a complex mixture of compounds one or more of which shows antibacterial activity and a target gene for that antibacterial compound. In the method according to this aspect of the invention, bacterial cells comprising a genomic library are contacted with the extract on a plating medium and cells that are resistant to the extract are selected, and the target gene that confers resistance to the extract is identified. The identification of the antibacterial compound in the extract is guided by exposing the sensitive and resistant cells to various fractions of the extract.

As used herein, the cells can include any bacterial cell including, but not limited to, Escherichia coli, Bacillus spp., Streptomyces coelicolor, Haemophilus influenzae, Staphylococcus aureus, Pseudomonal aeruginosa, or other bacterial species for which a plasmid DNA library can be constructed. Additionally, the cells can include any fungal or parasitic cell including, but not limited to Sacchoromyces cerevisiae, Schizosacchoromyces pombe, Neurospora crassa, Candida albicans, Trypanosoma spp., Giardia spp., Amoeba spp., Plasmodium spp. or other fungal or protozoan species for which a multicopy library can be constructed.

As used herein, the plating medium is any capable of supporting bacterial cell growth. Such plating mediums include, but are not limited to, LB-agar, Muller-Hinton agar, blood agar, or other medium standard in the art.

The term “target gene” is used herein to describe any gene capable of suppressing the activity of an antibiotic with antibacterial, antifungal or antiparasitic activities. The target gene can be identified through any means known to one skilled in the art. For example, plasmids from the cells that were resistant to the extract can be isolated and the insert sequenced. The resulting sequence can be then checked against a database of known sequences, for example, a BLAST search can be performed to determine the actual gene in the insert and its function in the cell if it is known. The target gene can also be validated by constructing a chromosomal deletion or replacement by standard methods of molecular biology. Target genes that encode proteins essential for cell viability or required for cell infection are the most interesting targets.

As used herein, the term “extract” refers to any natural product extract including, but not limited to, a natural plant extract, or extract prepared from marine, fungal, bacterial or mammalian sources. Preferably the extract will comprise a complex mixture of compounds one or more of which shows antibacterial activity. The term “compound” is used herein to describe any natural or synthetic compounds or organic small molecule compounds. To identify compounds in the extracts which have identified interesting targets, the extracts are fractionated and identification of the antibacterial compound is guided by exposing the sensitive and resistant cells to various fractions of the extract by the use of NMR and MS.

The terms “antibacterial compound” and “antibiotic” are used herein to describe a compound or composition which has antimicrobial, bacteriostatic, or bactericidal activity.

Antibacterial compounds that are identified through the method described herein can be then subjected to chemical modification via (semi)synthesis to identify further related compounds. Chemical modification can be guided by computer-aided drug design and automated organic synthesis to allow thousands of compounds (a library) of systematic variants of a parent chemical structure to be produced in parallel by synthesizing structurally related analogs and analyzing them for binding to the target molecule. Thus, millions of new compounds designed to inhibit a target can now be created in a relatively short time.

Many of the target genes selected from the E. coli genomic DNA library have shown the presence of MDR (Multi-Drug Resistance) inserts. These are usually direct pumps or transcriptional regulators of the pumps themselves. These types of inserts confer resistance, yet this resistance is only due to an efflux pump and are therefore not of primary interest. However, the results have demonstrated the high level of similarity in MDR components selected against differing cytotoxic compounds. An example of which is the EmrE gene, which codes for a transporter protein from the SMR (small Multi-Resistant) family. The sequence for the EmrE gene was able confer resistance to three unrelated compounds which gives credence to the idea that it is only acting as a pump. Four extracts have selected plasmids with genes encoded proteins for Mar operon (marA, marR, Rob). Although these types of results are not optimal, they do elucidate the mechanisms of drug resistance nicely. These methods described herein can be used to identify compounds that inhibit these pumps, and thus can potentiate antibiotics, thereby increasing their effectiveness.

However, not all the genes identified correspond to MDR pumps. At least 10 genes which code for proteins involved in E. coli metabolism have been identified and are potential new drug targets. These include L13 ribosomal protein, acetohydroxybutanoate synthase, glucokinase, glutamate decarboxylase, recombinase, acyl-coA-dehydrogenase, cAMP phosphodiesterase, and serine protease. Each has been identified in parallel with an inhibitor (or natural products extract containing an inhibitor).

IlvM is a member of IlvGMEDA operon, encoding enzymes involved in biosynthesis of branched-chain amino acids. This biosynthesis consists of several enzymatic steps that lead from pyruvate and α-ketobutyrate to valine and isoleucine and from α-ketovalerate to leucine. The IlvM gene encodes one subunit of the enzyme acetohydroxybutanoate synthase/acetolactate synthase, which is responsible for two enzymatic reactions and has a complex regulatory pathway. There are strong functional similarities among all enzymes in this family from all bacterial species. However, analogs of such enzymes are not found in mammals, therefore inhibitors of this pathway should be non-cytotoxic for mammalian cells.

Another prokaryotic only enzyme selected is the product of the cpdA gene. This gene functions as a c-AMP-phosphodiesterase. Enzymes of this family are well known as the key enzymes for different regulatory pathways. However, c-AMP-phosphodiasterase III, encoded by the cpdA gene, presents a unique class. Members of this class are widespread among bacterial species and have a conserved structure. BLAST searching has shown especially high homologies of the E. coli enzyme with Pasteurella multocida, Vibrio cholerae and Pseudomonas aeruginosa. However, this class of phosphodiesterases does not exist in eukaryotic organisms.

Other selections yielded two suppressor loci (rplM and YhiX). These encode an L13 ribosome protein and a Gad X transcriptional regulator of Gad operon, respectively. The functionality of these proteins has not been well characterized. However, indirect observations show that they are also important for cell survival and might be used as target candidates for new antibiotic research. Both of them have been shown to participate in stress response of bacterial cells. The L13 protein has been shown to bind with domain V of 23S rRNA, close to PTC. It participates in the formation of the transcriptional complex. Disruption of this protein leads to a breakdown of the ribosome assembly and maturation of 16S rRNA.

GadX belongs to the AraC/XylS family of bacterial transcriptional factors, known to be activators of such important cellular functions as sugar catabolism, as well as responses to stress and virulence. The gene product is part of the Gad operon and for a long time it was considered a transcriptional regulator of the glutamine decarboxylase system. This system is very important for a cells survival in aggressive environments. It is thought to reduce their intracellular pH by translocating charge across the cytoplasmic membrane and to provide metabolic energy to the microorganism. However, new experimental data have demonstrated that GadX has a wider spectrum of activity. Overexpression of GadX induces the expression of proteins from the Gad operon. New experiments have shown that GadX can also induce the expression of virulent bacterial factor PerA, osmC protein (responsible for adaptation at high osmolarity), and different proteases and chaperons (ion, ycgC, yehA and yhcA). It can also be involved in the expression of proteins, such as in the biosynthesis of aspargine (Asn) and glutamine (glnH, glnK).

In a second aspect, the invention provides a method for using a complex mixture of compounds one or more of which shows antibacterial activity for the parallel identification of an antibacterial compound and a target gene for that antibacterial compound. In the method according to this aspect of the invention, bacterial cells comprising a genomic library are contacted with the complex mixture of compounds on a plating medium and cells that are resistant to the complex mixture of compounds are selected, and the target gene that confers resistance to the complex mixture of compounds is identified. The mixture is fractionated and the identification of the antibacterial compound in the complex mixture of compounds is guided by exposing the sensitive and resistant cells to various fractions of the mixture. All definitions are as described above.

As used herein, a complex mixture of compounds refers to any mixture of synthetic and/or natural compounds or organic small molecule compounds or product libraries of compounds.

In a third aspect, the invention provides a method for identifying the mode of action of existing antibacterial agents. In the method according to this aspect of the invention, bacterial cells comprising a genomic library are contacted with one or more known antibacterial agents on a plating medium and cells that are resistant to the antibacterial agent are selected. The target gene that confers resistance to the extract is identified and the activity of the protein encoded by the target gene is determined. All definitions are as described above.

In a fourth aspect, the invention provides a method of finding novel antibacterial chemotypes that function by a specific mechanism of action. In the method according to this aspect of the invention, bacterial cells comprising an expression vector containing a known gene are contacted, on a plating medium, with an extract comprising a complex mixture of compounds one or more of which shows antibacterial activity. The extract is fractionated and the identification of the antibacterial compound in the extract is guided by exposing resistant cells to various fractions of the extract. All definitions are as described above.

The following examples are intended to further illustrate certain particularly preferred embodiments of the invention and are not intended to limit the scope of the invention.

### EXAMPLE 1

### Identification of Antibacterial Compounds from an Extract

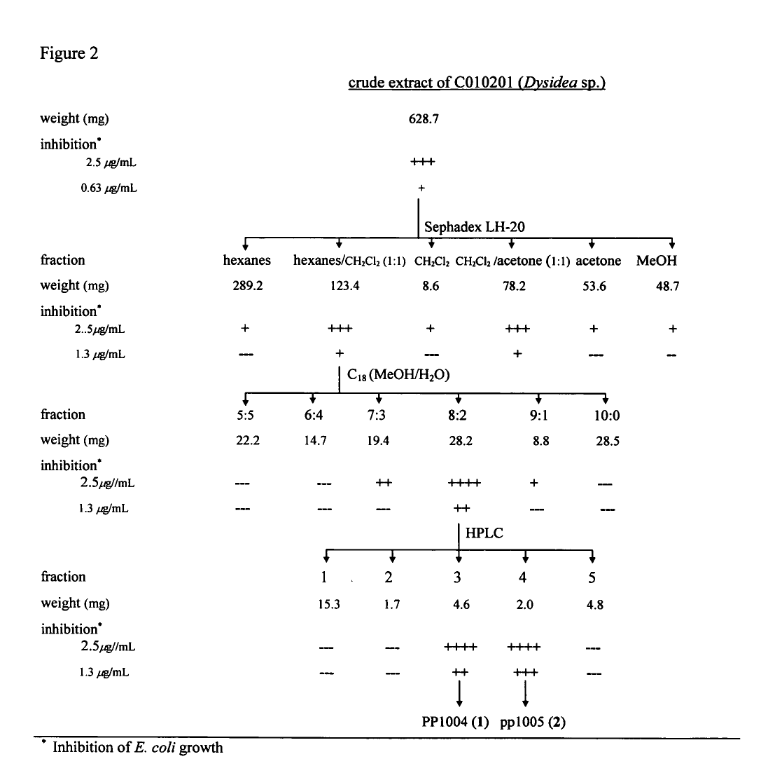

A marine extract, prepared from C010201 (Dysidea sp.), showed some possible activity toward certain targets. The crude extract (629 mg) was subjected initially to fractionation on a Sephadex LH-20 column (40 g), which was washed successively with hexanes, 1:1 hexanes-CH₂Cl₂, CH₂Cl₂, 1:1 CH₂Cl₂-acetone, acetone, and methanol (FIG. 2). The 1:1 hexanes-CH₂Cl₂ and 1:1 CH₂Cl₂-acetone fractions displayed the most inhibitory activity against E. coli growth. Further fractionation of the 1:1 hexanes-CH₂Cl₂ (123 mg) came on a C₁₈ open column, which was eluted successively with 5:5, 6:4, 7:3, 8:2, 9:1, and 10:0 MeOH—H₂O; this provided the strongest activity 8:2 MeOH—H₂O fraction. A purification of this fraction was applied to a reverse phase C₁₈ HPLC column (250×10 mm, 5 μm) which was washed with 60→95% MeCN in water over 35 min at a flow rate 4.5 mL/min (detected at 240 nm). This afforded two compounds identified, respectively, as 2-(2′,4′-dibromophenoxy)-3,4,5-tribromophenol (PP1004, 1), and 2-(2′,4′-dibromophenoxy)-4,5,6-tribromophenol (PP1005, 2), by comparison of their spectral data with literature data. Identification of targets for these compounds can be done in parallel as described below.

### EXAMPLE 2

### Parallel Identification of an Antibacterial and Its Target

E. coli DNA library was constructed by ligating a partial Sau 3A digest of total genomic DNA of E. coli K-12 (ATCC #25404) into pgem 3Z vector (Promega Corp.). The resultant pool of plasmids was transformed in E. coli strains DH5α (Invitrogen). The DNA library had a complexity of about 10⁵. The average insert size in the library was 2.0 kb with more than 90% of the transformed cells containing plasmid. This leads to a theoretical 200-fold coverage of the genome.

Selection of targets was carried out on LB-agar plates, supplemented with the cytotoxic extract of interest. DNA library and control cells (with vector, no insert) were applied on four plates with differing extract concentrations. Colonies located on the highest concentration of extract were then selected and tested on an X-GAL indicator plate. The ratio between white and blue colonies then determines the efficiency of selection. Appearance of blue colonies results in a false positive result. This could be explained as a possible host chromosomal mutation in the presence of the tested compound.

Plasmids from white colonies were purified and tested for their ability to confer resistance when retransformed into host E. coli strain. Plasmids which held up to this retransformation were then sent out for sequencing. The insert was sequenced from each end using both a T7 and an SP6 promoter primer. The resultant sequences were then used in BLAST searches to determine the actual genes in the insert. Sequences which contained full genes which were not multi-drug efflux pumps are then carried over to the next phase of the project. Extracts, which have shown interesting targets, were fractionated and inhibitor compounds were identified by the use of NMR and MS.

Inserts with multiple genes present are then separated by way of PCR. Each individual gene is inserted into a separate plasmid to determine its ability to confer resistance to the requisite extract. Using this method, we are able to determine which gene is responsible for the resistance to the extract, thereby narrowing down of search for its target. Further manipulation of the gene allows the sensitivity of the extract to be determined by way of an in-frame deletion of the protein as well as removal of the gene from the native E. coli.

What is claimed is:

1. A method for the parallel identification of (1) an antibacterial compound from an extract comprising a complex mixture of compounds one or more of which shows antibacterial activity and (2) a target gene for that antibacterial compound, the method comprising contacting bacterial cells comprising a genomic library with the extract on a plating medium and selecting cells that are resistant to the extract, identifying the target gene that confers resistance to the extract, guiding fractionation of the extract by exposing the sensitive and resistant cells to the various fractions, and thereby identifying the antibacterial compound in the extract.

2. The method according to claim 1, wherein the plating medium is selected from the group consisting of LB-agar, Muller-Hinton agar and blood agar.

3. The method according to claim 1, wherein the bacterial cells are selected from the groups consisting of E. coli, Bacillus spp., Streptomyces coelicolor, Haemophilus influenzae, Staphylococcus aureus and Pseudomonas aeruginosa.

4. A method for using a complex mixture of compounds one or more of which shows antibacterial activity for the parallel identification of (1) an antibacterial compound and (2) a target gene, the method comprising contacting bacterial cells comprising a genomic library with the complex mixture of compounds on a plating medium and selecting cells that are resistant to the complex mixture of compounds, identifying the target gene that confers resistance to the complex mixture of compounds, fractionating the complex mixture of compounds and exposing the resistant cells to the various fractions, and identifying the antibacterial compound in the complex mixture of compounds.

5. The method according to claim 4, wherein the plating medium is selected from the group consisting of LB-agar, Muller-Hinton agar and blood agar.

6. The method according to claim 4, wherein the bacterial cells are selected from the groups consisting of E. coli, Bacillus spp., Streptomyces coelicolor, Haemophilus influenzae, Staphylococcus aureus and Pseudomonas aeruginosa.

7. A method for identifying the mode of action of existing antibacterial agents, the method comprising contacting bacterial cells comprising a genomic library with one or more known antibacterial agents on a plating medium and selecting cells that are resistant to the antibacterial agent, identifying the target gene that confers resistance to the extract, and determining the activity of the protein encoded by the target gene.

8. A method for finding antibacterial chemotypes that function by a specific mechanism of action, the method comprising contacting, on a plating medium, bacterial cells comprising an expression vector comprising a known gene with an extract comprising a complex mixture of compounds one or more of which shows antibacterial activity and guiding fractionation of the extract by exposing the sensitive and resistant cells to the various fractions, and thereby identifying the antibacterial compound in the extract.
